(19) United States
(12) Patent Application Publication
(10) Pub. No.: US 2024/0270689 A1
(43) Pub. Date: Aug. 15, 2024

(54) PROCESS FOR PRODUCING BRIVARACETAM AND INTERMEDIATES THEREOF

(71) Applicant: Carnitech LLC, North Brunswick, NJ (US)

(72) Inventors: Songzhou Hu, Princeton, NJ (US); Zhen Song, Shanghai (CN)

(21) Appl. No.: 18/368,754

(22) Filed: Sep. 15, 2023

Related U.S. Application Data
(60) Provisional application No. 63/441,890, filed on Jan. 30, 2023.

(51) Int. Cl. C07D 207/12; C07C 229/76; C07C 233/36; C07C 255/63

(52) U.S. Cl. CPC C07D 207/12; C07C 229/76; C07C 233/36; C07C 255/63

(57) ABSTRACT

There is disclosed a process for the production of brivaracetam by converting an intermediate of the formula:

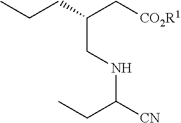

to an intermediate of the formula:

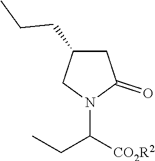

and then to brivaracetam; wherein R¹ and R² are each independently selected from the group consisting of hydrogen, alkali, alkaline earth metal, alkyl, and a mixture thereof.

### CROSS REFERENCE

This application claims the benefit under 35 U.S.C. § 119(e) from U.S. Provisional Application No. 63/441,890, filed on Jan. 30, 2023, the contents of which are incorporated by reference herein.

### FIELD OF THE INVENTION

This invention relates to a process for the production of brivaracetam and intermediates thereof.

### BACKGROUND OF THE INVENTION

Brivaracetam belongs to a class of racetam medications that is used to treat epilepsy and related central nervous system disorders. Brivaracetam is chemically (2S)-2-[(4R)-2-oxo-4-propylpyrrolidin-yl]butanamide of the following structure:

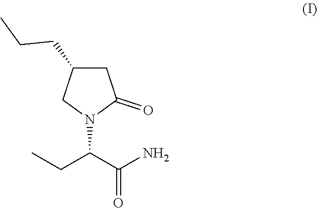

There are numerous methods for the synthesis of brivaracetam. Gayke et al have exhaustively reviewed methods for the synthesis of brivareacetam in Synthetic Approaches towards the Synthesis of Brivaracetam: An Antiepileptic Drug (M. Gayke, H. Narode, G. Eppa, R. S. Bhosale, and J. S. Yadav, ACS Omega, 2022, Vol. 7, pp 2486-2503), which is incorporated herein in entirety by reference.

The presence of two chiral centers in brivaracetam creates special challenge in processes for industrial production. If the (2S) chiral carbon is acquired from commercially available L-2-aminobutanamide, the second (4R) chiral carbon needs to be constructed from derivatives of an enantiomerically pure (4R)-propyl-butyrolactone, which is not readily available and difficult to produce. On the other hand, if the (4R) chiral carbon is derived from (4R)-propyl-2-oxo-pyrrolidine, (2R)-halobutyric acid derivative must be used to furnish the second chiral carbon. However, (2R)-halobutyric acid derivative is not only commercially unavailable, but also extremely obnoxious, posing environmental and occupational problems. Moreover, even if the two pure chiral building blocks are used to construct brivaracetam, it is still difficult to obtain optically pure brivaracetam, because the chiral central in L-2-aminobutanamide or (2R)-halobutyric acid is susceptible to racemization during the reaction.

It has been known that when one of the two chiral centers is used to induce the other chiral center, an impure product is invariably obtained. Extensive purification, including liquid chromatograph, will be required to yield an optically pure brivaracetam. After the purification, the unwanted optical isomer is wasted, since racemization after optical resolution and enrichment is not known for brivaracetam, especially for the inactive (4R)-carbon of 2-oxo-pyrrolidine moiety.

It is the objective of the present invention to overcome these inherent disadvantages in the processes for preparing brivaracetam and to disclose a process for preparing brivaracetam and intermediates thereof from readily available and easily prepared starting materials. The process according to the present invention is concise, constructs all necessary structural elements in a cascade of reactions and reaches brivaracetam in far fewer steps than prior art processes. Moreover, all unwanted optical isomer can be readily racemized and recycled in the process according to the present invention.

### SUMMARY OF THE INVENTION

This invention relates to a process for the production of brivaracetam from a novel intermediate of the following structure (II):

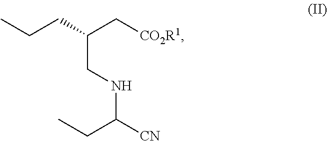

wherein R¹ is hydrogen, alkyl, alkali, or alkaline earth metal, and wherein the alkali is lithium, sodium, potassium or a mixture thereof, wherein the alkaline earth metal is magnesium, calcium, barium, or a mixture thereof, and wherein the alkyl refers to C₁-C₁₂ of a straight, cyclic, or branched chain alkane radical containing from 1 to 12 carbon atoms, preferably the alkyl is C₁-C₄.

The invention is accomplished by converting the intermediate of formula (II) into an intermediate of formula (V):

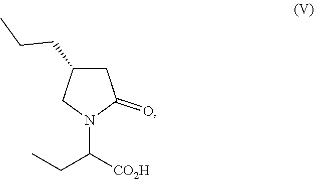

its optical resolution into an intermediate of formula (VI):

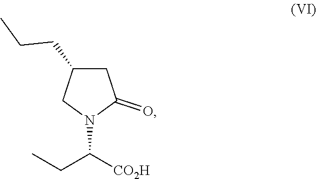

and the preparation of brivaracetam thereafter.

### DETAILED DESCRIPTION OF THE INVENTION

### Definitions

The term “alkyl” refers to a straight, branched chain, or cyclic alkane (hydrocarbon) radical containing from 1 to 12 carbon atoms. Exemplary “alkyl” groups include methyl, ethyl, propyl, isopropyl, cyclopropyl, n-butyl, t-butyl, isobutyl, cyclobutyl, cyclopropylmethyl, pentyl, isopentyl, hexyl, isohexyl, heptyl, 4,4-dimethylpentyl, octyl, 2,2,4-trimethylpentyl, nonyl, decyl, undecyl, dodecyl, and the like. The term “C₁-C₈ alkyl” refers to a straight, cyclic, or branched chain alkane radical containing from 1 to 8 carbon atoms, such as methyl, ethyl, propyl, isopropyl, cyclopropyl, n-butyl, t-butyl, isobutyl, cyclobutyl, cyclopropylmethyl, pentyl, isopentyl, hexyl, isohexyl, heptyl, 4,4-dimethylpentyl, and octyl.

The compounds of the present invention may form salts which are also within the scope of this invention. Reference to compounds of the formula (II) through (XII) herein is understood to include reference to salts thereof, unless otherwise indicated. The term “salt(s)”, as employed herein, denotes acidic and/or basic salts formed with inorganic and/or organic acids and bases.

The compounds of the present invention may form salts with a variety of organic and inorganic acids. Exemplary acid addition salts include hydrochloric acid, hydrobromic acid, sulfuric acid, nitric acid, phosphoric acid, alkanoic acids, alkylsulfonic acids, aromatic sulfonic acids, isethionic acid, and the like.

The compounds of the present invention may also form salts with a variety of organic and inorganic bases. Exemplary basic salts include ammonium salts, alkali metal salts such as lithium, sodium, potassium salts, alkaline earth metal salts such as calcium and magnesium salts, salts with organic bases (for example, organic amines) and salts with amino acids such as arginine, lysine and the like.

All stereoisomers of the present compounds (for example, those which may exist due to asymmetric carbons on various substituents), include enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The individual optical isomers can be obtained from the racemates by any suitable method, including without limitation, conventional methods, such as, for example, salt formation with an optically active acid or base followed by crystallization, or biocatalytic methods, for example, selective hydrolysis with a lipase.

### DESCRIPTION OF THE INVENTION

The present invention relates to processes for producing brivaracetam from N-[(2R)-propyl-3-carboxy]-2-aminobutyronitrile of formula (II):

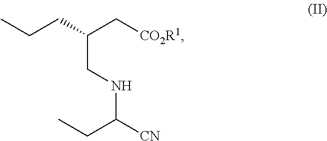

wherein R¹ is hydrogen, alkyl, alkali, or alkaline earth metal, and wherein the alkali is lithium, sodium, potassium or a mixture thereof, wherein the alkaline earth metal is magnesium, calcium, barium, or a mixture thereof, and wherein the alkyl refers to C₁-C₁₂ of a straight, cyclic, or branched chain alkane radical containing from 1 to 12 carbon atoms, preferably the alkyl is C₁-C₄.

The first embodiment of the process according to the present invention to produce brivaracetam of formula (I) can be illustrated in the following reaction scheme:

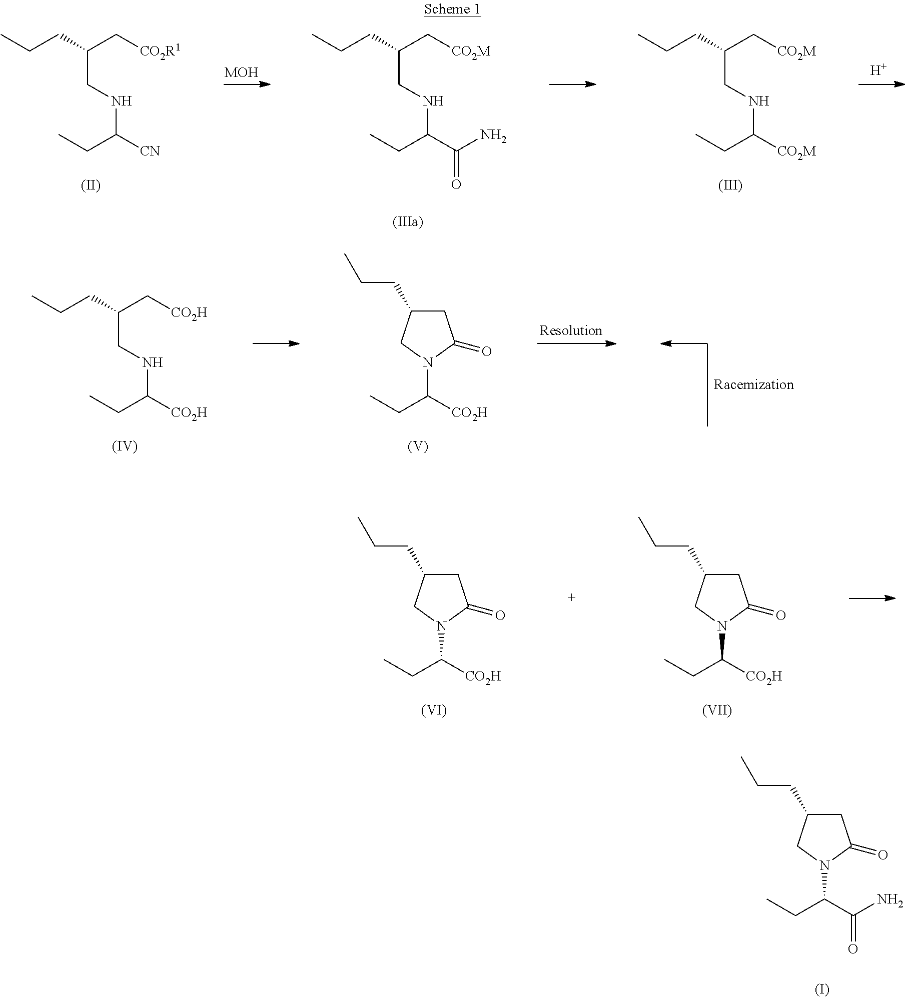

In this preferred embodiment, the intermediate of formula (II) is converted to an intermediate of formula (III) by reacting with a base, denoted as MOH, wherein M is an alkali or an alkaline earth metal or their mixture. The alkali is lithium, sodium, potassium, or a mixture. The alkaline earth metal is magnesium, calcium, barium, or a mixture.

Preferably, the intermediate of formula (II) is first hydrated to an amide of formula (IIIa) in the presence of a catalyst. A suitable catalyst is selected from the group consisting of lithium hydroxide, sodium hydroxide, potassium hydroxide, cesium hydroxide, magnesium hydroxide, calcium hydroxide, barium hydroxide, magnesium oxide, calcium oxide, barium oxide, and a mixture of two or more thereof. Preferably, the catalyst is the same as the one used for the hydrolysis to the carboxylate of formula (III). More preferably, an alkali hydroxide is used. Most preferably, sodium hydroxide is used.

The temperature for the hydration of cyano group can be from 0° C. to 60° C., preferably from 5° C. to 40° C., more preferably, from 10° C. to 35° C., most preferably, from 15° C. to 30° C.

It has been found that when the reaction temperature is higher then 60° C., the yield is significantly decreased.

In this aspect of the first embodiment of the present invention, the amide group in the intermediate of formula (IIIa) is then hydrolyzed to the carboxylate of formula (III) by using an alkali hydroxide or an alkaline earth metal hydroxide or an alkaline earth metal oxide.

A suitable hydroxide is selected from the group consisting of lithium hydroxide, sodium hydroxide, potassium hydroxide, cesium hydroxide, magnesium hydroxide, calcium hydroxide, barium hydroxide, magnesium oxide, calcium oxide, barium oxide, and a mixture of two or more thereof. The resulting carboxylate salt is acidified to form an intermediate of formula (IV) by adding an acid. A suitable acid is selected from the group consisting of hydrochloric acid, sulfuric acid, nitric acid, phosphoric acid, hydrobromic acid, lower alkanoic acid, alkylsulfonic acid, aromatic sulfonic acid, and a mixture thereof.

The intermediate of formula (IV) is then cyclized to form (2RS)-2-[(4R)-2-oxo-4-propylpyrrolidin-yl]butyric acid of formula (V), a key and novel intermediate to brivaracetam in the process according to present invention.

In one method to prepare the key intermediate of formula (V), the intermediate of formula (IV) is heated to melt while removing the water formed during the cyclization. After no more water is released, the cyclization is complete. The product can be recrystallized from water or used for the next step of the process.

In another method to prepare the key intermediate of formula (V), the intermediate of formula (IV) is suspended in a solvent of a boiling point of at least 100° C. and heated to reflux to perform the cyclization reaction while removing the formed water. A suitable solvent is selected from the group consisting of toluene, xylenes, trimethybenzenes, cumene, cymene, dimethylformamide, dimethylacetamide, N-methylpyrrolidinone, dimethylsulfoxide, C₄-C₁₀ alcohols, and C₂-C₈ alkanoic acids.

In a further method to prepare the key intermediate of formula (V), the intermediate of formula (IV) is suspended in water and is heated to a temperature from 100° C. to 200° C. under autogenous pressure to perform the cyclization reaction of (IV) to (V). Preferably, the reaction temperature is from 110° C. to 180° C. More preferably, from 120° C. to 160° C. And most preferably, from 130° C. to 150° C. After the reaction, the product precipitates from the aqueous solution upon cooling. After separation of the product of formula (V) by a solid-liquid separation, the mother liquor can be used to suspend a new batch of the intermediate (IV) and to perform the cyclization reaction. This cyclic process of cyclization of the intermediate (IV) results in a quantitative yield of the key intermediate (V) without using any additional reagent.

It is particularly advantageous to carry out all steps of reactions leading to the key intermediate of formula (V) in a cascade of reactions, without the tedious isolation of any intermediate. This cascade process of multiple reactions according to the present invention results in a concise process for preparing brivaracetam.

The compound of formula (II) can be prepared efficiently in a process comprised of reacting propionaldehyde, a source of cyanide, and (3R)-propyl gamma-aminobutyric acid derivative of formula (VIII) in a reaction as described in the following scheme:

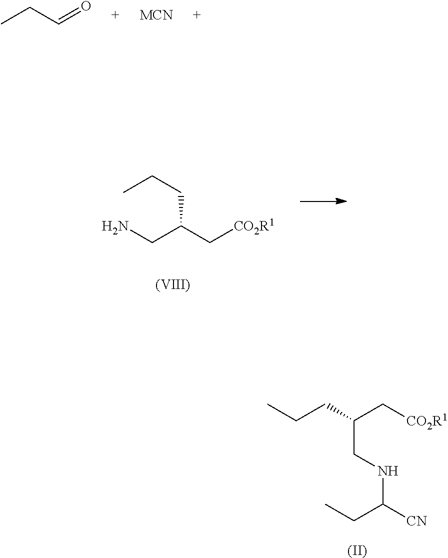

wherein MCN represents a source of cyanide such as alkali cyanide, alkaline earth metal cyanide, zinc cyanide, or hydrogen cyanide, wherein R¹ is hydrogen, alkyl, alkali, or alkaline earth metal, and wherein the alkali is lithium, sodium, potassium or a mixture thereof, wherein the alkaline earth metal is magnesium, calcium, barium, or a mixture thereof, and wherein the alkyl refers to C₁-C₁₂ of a straight, cyclic, or branched chain alkane radical containing from 1 to 12 carbon atoms, preferably the alkyl is C₁-C₄.

The (3R)-propyl gamma-aminobutyric acid derivative of formula (VIII) can be prepared by methods known in prior art. It can be used as pure product. It can also be used as a resultant product of previous reaction without further isolation and purification. The optical purity of (3R)-propyl gamma-aminobutyric acid derivative of formula (VIII) is at least 90%. Preferably, at least 95%; more preferably, at least 97%; most preferably, at least 99%.

The reaction to prepare the intermediate of formula (II) is carried out in an aqueous solution and optionally in the presence of an organic solvent. A suitable solvent is water-soluble and is selected from the group consisting of methanol, ethanol, propanol, isopropanol, butanol, isobutanol, tert-butanol, tetrahydrofuran, dioxane, methoxyethanol, ethoxyethanol, and a mixture thereof. Preferably, the reaction is carried in an aqueous solution.

The method of preparing the intermediate of formula (II) according to the present invention also comprises reacting the Schiff base of the following formula:

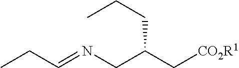

with a source of cyanide, wherein the Schiff base may be prepared in situ, comprising reacting propionaldehyde with the intermediate of formula (VIII).

The method of preparing the compound of formula (II) according to the present invention further comprises reacting the cyanohydrin of propionaldehyde of the following formula:

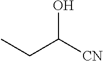

with the compound of formula (VIII). The cyanohydrin of propionaldehyde can be prepared from the reaction of propionaldehyde and a source of cyanide such as such as alkali cyanide, alkaline earth metal cyanide, zinc cyanide, or hydrogen cyanide. The cyanohydrin can be prepared in situ and used as such or isolated as a pure product and then used.

After its preparation, the compound of formula (II) may be isolated from the reaction mixture. On the other hand, it may also be used as such for subsequent reaction.

In the first embodiment of the present invention, a base is used to hydrolyze the cyano group in the compound of formula (II). The base is selected from the group of alkali hydroxide, alkali carbonate, alkaline earth metal hydroxide, and a mixture thereof. The alkaline solution is then acidified with an acid to yield a compound of formula (IV), which is then cyclized to the key compound of formula (V). A suitable acid is selected from the group consisting of hydrochloric acid, sulfuric acid, nitric acid, phosphoric acid, hydrobromic acid, lower alkanoic acid, alkylsulfonic acid, aromatic sulfonic acid, and a mixture thereof.

The compounds of formula (III) and (IV) may be isolated and purified. Preferably, they are not isolated, but used directly in a cascade of reactions to the key compound of formula (V). The overall molar yield of the key intermediate of formula (V) in this cascade of reactions is at least 50%, particularly more than 70%, more particularly more than 80%, most particularly more than 85%.

In the other aspect of the first embodiment of the present invention, the key intermediate of formula (IV) is prepared directly by reacting the compound of formula (II) with an acid to hydrolyze the nitrile group. A suitable acid is selected from the group consisting of sulfuric acid, hydrochloric acid, nitric acid, phosphoric acid, hydrobromic acid, alkylsulfonic acid, aromatic sulfonic acid, and a mixture of two or more thereof. Preferably, sulfuric acid is used to transform the compound of formula (II) to the intermediate of formula (IV).

The racemic compound of formula (V) is then optically resolved by using an optically active resolving agent. A suitable resolving agent can be selected from those listed in David Kozma, CRC Handbook of Optical Resolution via Diastereomeric Salt Formation, 2002, CRC Press, which is incorporated herein in its entirety.

Among the resolving agents of chiral amines, dehydroabietylamine was found to form a crystalline salt that can be used to resolve the compound of formula (V) to the compound of formula (VI). The salt is of the following structure:

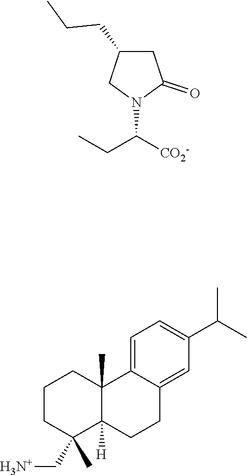

After optical resolution of the (2S)-2-[(4R)-2-oxo-4-propylpyrrolidin-yl]butyric acid of formula (VI), the (2R)-2-[(4R)-2-oxo-4-propylpyrrolidin-yl]butyric acid of formula (VII) is then racemized to (2RS)-2-[(4R)-2-oxo-4-propylpyrrolidin-yl] butyric acid of formula (V) and subjected to optical resolution again. The racemization reaction of (2R)-2-[(4R)-2-oxo-4-propylpyrrolidin-yl] butyric acid of formula (VII) may be carried out in a solution of alkali hydroxide.

This cyclic method of resolution and racemization can yield the (2S)(4R)-enantiomer in a yield of more than 50%, particularly more than 75% to nearly quantitative. Hence, this high efficiency in the process according to the present invention results in no loss of valuable late-stage material, a distinct advantage over prior art methods.

The optically pure (2S)-2-[(4R)-2-oxo-4-propylpyrrolidin-yl]butyric acid of formula (VI), can be readily converted to brivaracetam of formula (I) by methods known for one skilled in the art for the conversion of carboxylic acid to amide.

In the second embodiment of the present invention to prepare brivaracetam, the intermediate of formula (II) is converted to an intermediate of formula (IV) according to methods described in the first embodiment of the present invention and then to an ester of formula (VI) by reacting the intermediate of formula (IV) with an alcohol of the formula R¹OH, wherein R¹ is a C₁-C₁₂ alkyl group, preferably, C₁-C₄ alkyl group. This racemic ester of formula (IX) is optically resolved by using a hydrolytic enzyme to selectively hydrolyze the ester to an optically active acid (VI), which is used to prepare brivaracetam in a process comprising the steps according to the following reaction scheme:

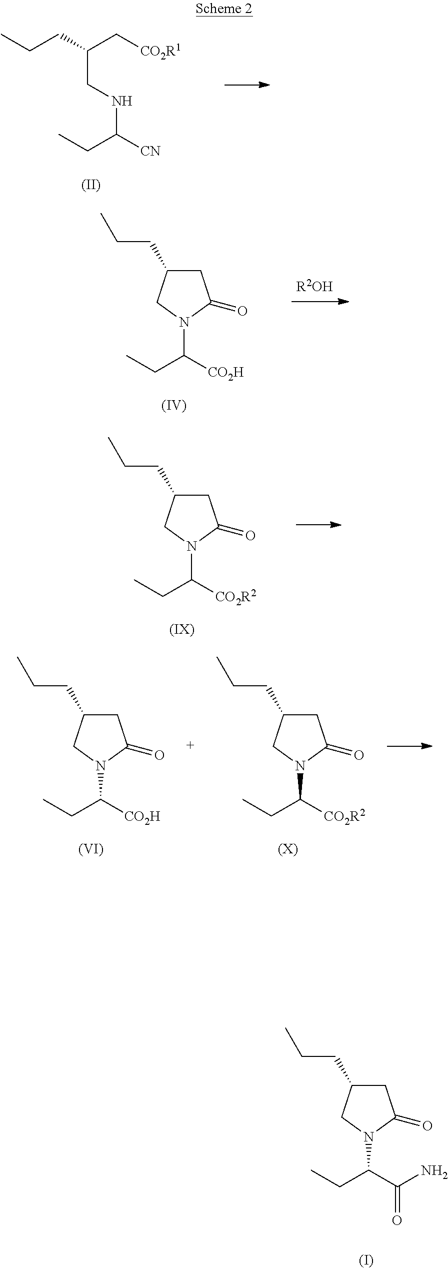

wherein R² is a C₁-C₁₂ alkyl group. Preferably, R² is a C₁-C₄ alkyl group.

A suitable enzyme is alkaline protease, for example, the commercially available Alcalase. This enzyme is found to selectively hydrolyze the ester of formula (IX) to form the compound of formula (VI). The optically purity of formula (IV) is found to be greater than 90%. This impure product may be purified by recrystallization from a solvent. Preferably, this product is purified by forming a salt with dehydroabietylamine. After purification, the purity for the intermediate of formula (IV) can be increased to at least 98%, preferably to greater than 99%.

After the enzymatic hydrolysis, the compound of formula (X) may be racemized in the presence of a base. A suitable base can be selected from the group consisting of alkali alkoxide, alkali hydride, alkali amide, and a mixture thereof. A particularly suitable base is sodium methoxide. On the other hand, the compound of formula (X) may be hydrolyzed to a compound of formula (VII) and then racemized in a solution of alkali hydroxide.

In the third embodiment of the present invention, the compound of formula (II) is first converted to a diester of formula (VIII), which is then cyclized to the ester of formula (VI). The ester is then selectively hydrolyzed by a hydrolytic enzyme to an optically active acid. Brivaracetam is prepared from the acid of formula (VI).

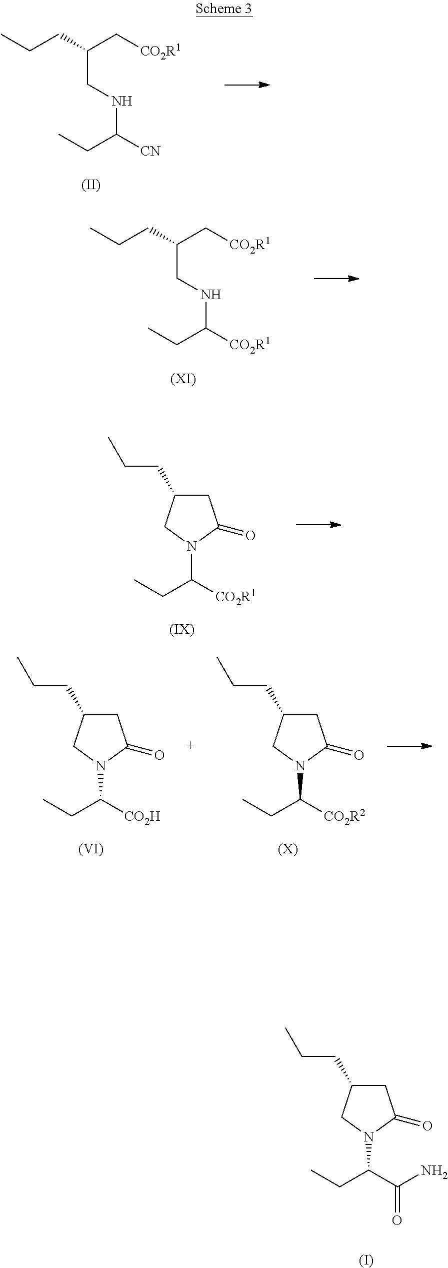

wherein R¹ is a C₁-C₁₂ alkyl group. Preferably, R¹ is a C₁-C₄ alkyl group.

The formation of the diester of formula (XI) is performed by reacting the compound of formula (II) with an alcohol R¹OH in the presence of an acid, wherein R¹ is a C₁-C₁₂ alkyl group, preferably, C₁-C₄ alkyl group. A suitable acid is selected from the group consisting of hydrochloric acid, hydrobromic acid, sulfuric acid, nitric acid, alkylsulfonic acid, and arylsulfonic acid. Preferably, sulfuric acid is used. The formed diester may be isolated or used directly for the cyclization to form the intermediate of formula (IX). The cyclization reaction is carried out with a base and optionally in the presence of a phase transfer catalyst. A suitable base is selected from the group consisting of alkali hydroxide, alkali carbonate, alkali phosphate, alkali sulfite, alkaline earth metal hydroxide, alkaline earth metal oxide, or a mixture thereof. A suitable phase transfer catalyst is selected from the group consisting of quaternary ammonium salts and quaternary phosphonium salts. Preferably, benzyltriethylammonium chloride or tetrabutylammonium bromide is used.

In the fourth embodiment of the present invention, the intermediate of formula (II) is cyclized to an intermediate of formula (XII):

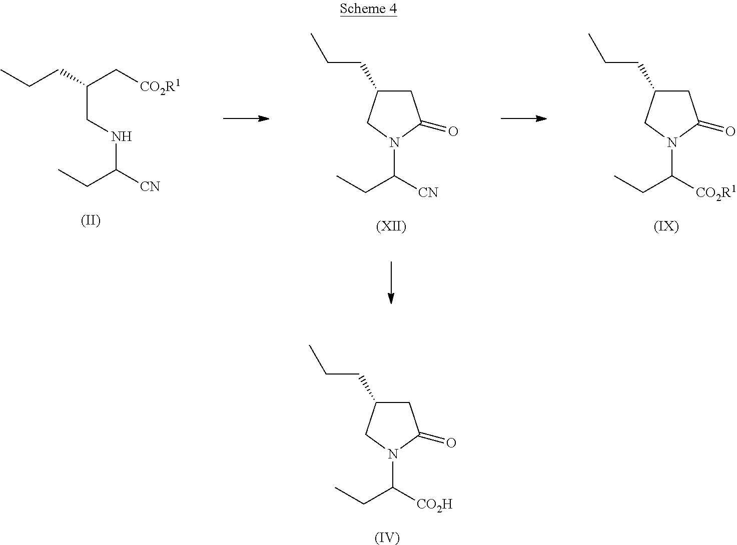

wherein R¹ is a C₁-C₁₂ alkyl group. Preferably, R¹ is a C₁-C₄ alkyl group.

The formation of the intermediate of formula (XII) is performed with a base and optionally in the presence of a phase transfer catalyst. A suitable base is selected from the group consisting of alkali hydroxide, alkali carbonate, alkali phosphate, alkali sulfite, alkaline earth metal hydroxide, alkaline earth metal oxide, or a mixture thereof. A suitable phase transfer catalyst is selected from the group consisting of quaternary ammonium salts and quaternary phosphonium salts. Preferably, benzyltriethylammonium chloride or tetrabutylammonium bromide is used.

The nitrile group in the compound of (XII) can be hydrolyzed to a carboxylic acid to obtain the intermediate of formula (IV) or an ester to form the intermediate of formula (IX). These two intermediates are then optically resolved and converted to brivaracetam according to methods described in the first and second embodiments of the present invention.

### EXAMPLES

The following examples will illustrate the practice of this invention but are not intended to limit its scope.

### Example 1

To a solution comprised of 200 mL of water, 10 g of sodium cyanide, and 29 g of (3R)-propyl gamma butyric acid in a 1 L flask was dropwise added 12 g of propionaldehyde while maintaining the temperature below 25° C. After being stirred for 2 hours at room temperature, the solution was found to be comprised of 2-(RS)-N-(2R-propyl-carboxylpropyl)-aminobutyonitrile of formula (II). The LC-MS⁺¹ is 213. The solution was used without further purification.

### Example 2

To a solution comprised of 50 mL of water, 10 g of sodium cyanide, and 29 g of (3R)-propyl gamma butyric acid in a 1 L flask was dropwise added 12 g of propionaldehyde while maintaining the temperature below 25° C. After the solution was stirred at room temperature for 2 hours, 25 g of 30% sodium hydroxide was added. The temperature was observed to rise to 30° C. to form 2-(RS)-N-(2R-propyl-carboxylpropyl)-aminobutanamide of formula (IIIa). The LC-MS⁺¹ is 231.

### Example 3

To a solution comprised of 200 mL of water, 10 g of sodium cyanide, and 29 g of (3R)-propyl gamma butyric acid in a 1 L flask was dropwise added 12 g of propionaldehyde while maintaining the temperature below 25° C. After being stirred for 2 hours at room temperature, to the solution was added 24 g of 50% sodium hydroxide at once. The solution was stirred for 4 hours and then heated to reflux for 4 hours. The solution was then cooled to room temperature and mixed with 50 mL of 30% hydrochloric acid to obtained an intermediate of formula (VI). The LC-MS⁺¹ is 232.

### Example 4

23 g of the intermediate of formula (VI) was suspended in 100 mL of water in a pressure bottle and heated to 130° C. for 8 hours. After cooling to room temperature, crystalline solid was formed and filtered to yield 17 g of (2RS)-2-[(4R)-2-oxo-4-propylpyrrolidin-1-yl]butanoic acid of formula (V). The LC-MS⁺¹ is 214.

### Example 5

To 30 mL of acetonitrile was added 4.2 g of white recrystallized of (2RS)-2-[(4R)-2-oxo-4-propylpyrrolidin-1-yl]butanoic acid of formula (V) and 6.1 g of dehydroabietylamine. The mixture was briefly heated to 60° C. to obtain a clear solution. Upon cooling to room temperature, crystalline salt formed. The crystalline solid was filtered and recrystallized twice from 15 mL of ethyl acetate. The product is the dehydroabietylamine salt of (2S)-2-[(4R)-2-oxo-4-propylpyrrolidin-1-yl]butanoic acid of formula (VI), in which the optical purity of the compound of formula (VI) is 99%.

The salt was dissociated in 20 mL of deionized water containing 1 g of sodium hydroxide. After the dehydroabietylamine was removed by extraction with toluene two times, the aqueous solution was acidified to pH 2-3 with sulfuric acid. The mixture was cooled on ice and the crystals were filtered off to obtain (2S)-2-[(4R)-2-oxo-4-propylpyrrolidin-1-yl]butanoic acid of formula (VI).

1 g of the (2S)-2-[(4R)-2-oxo-4-propylpyrrolidin-1-yl]butanoic acid of formula (VI) was dissolved in 30 mL of anhydrous methanol containing 0.1 g of p-toluenesulfonic acid. After the solution was refluxing 2 hours to form the methyl ester, the solution was concentrated under vacuum to obtain an oil residue, to which 20 mL of 25% ammonia solution was added. The solution was kept at room temperature for 16 hours. After the excess ammonia and methanol were removed under vacuum, 50 m mL of dichloromethane was used to extract the product. After removal of dichloromethane, 0.7 g of a white solid product was obtained as (2S)-2-[(4R)-2-oxo-4-propylpyrrolidin-yl]butanamide of formula (I). The LC-MS⁺¹ is 213.

### Example 6

To a flask were added 30 mL of methanol, 2 g of (2RS)-2-[(4R)-2-oxo-4-propylpyrrolidin-1-yl]butanoic acid of formula (V), and 0.1 g of p-toluenesulfonic acid monohydrate. The solution was refluxed for 2 hours to form the methyl ester (2RS)-2-[(4R)-2-oxo-4-propylpyrrolidin-1-yl]butanoic acid of formula (IX). The methanol was removed under vacuum to yield an oily residue. To the oily residue were added 20 mL of water containing 1.0 g of sodium bicarbonate and 0.6 g of Alcalase. The mixture was vigorously stirred at room temperature for 8 hours. To the mixture was added 10 mL of methylene chloride to remove the unreacted methyl ester. The aqueous solution was adjusted to pH 1 by adding hydrochloric acid to yield 0.7 g of (2S)-2-[(4R)-2-oxo-4-propylpyrrolidin-1-yl]butanoic acid of formula (VI). The optical purity is 91%.

It will be understood that the foregoing examples and explanation are for illustrative purposes only and that in view of the instant disclosure various modifications of the present invention will be self-evident to those skilled in the art. Such modifications are to be included within the spirit and purview of this application and the scope of the appended claims.

What is claimed is:

1. A process for the production of brivaracetam of formula (I): 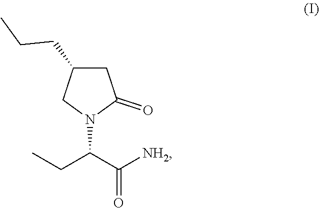
comprising:
(a) converting an intermediate of formula (II) to an intermediate of formula (IX) 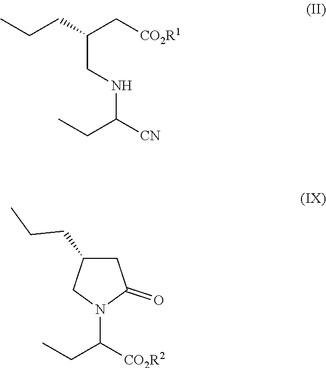
wherein R¹ and R² are each hydrogen, alkyl, alkali, or alkaline earth metal; wherein the alkali is lithium, sodium, potassium or a mixture thereof, wherein the alkaline earth metal is magnesium, calcium, barium, or a mixture thereof; and wherein the alkyl refers to C₁-C₁₂ of a straight, cyclic, or branched chain alkane radical containing from 1 to 12 carbon atoms,
(b) resolving the intermediate of formula (IX) to an intermediate of formula (X) 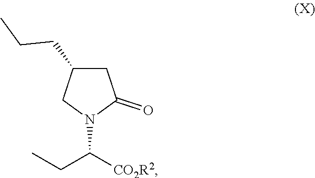
and
(c) converting the intermediate of formula (X) into brivaracetam of formula (I).

2. The process according to claim 1, wherein the intermediate of formula (II) is produced in a process comprising reacting propionaldehyde, a source of cyanide, and a primary amine of the following formula: 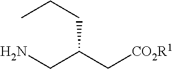
in an aqueous solution and optionally in the presence of a solvent, wherein R¹ is selected from the group consisting of hydrogen, alkali, alkaline earth metal, alkyl, and a mixture thereof; wherein the source of cyanide is alkali cyanide, alkaline earth metal cyanide, zinc cyanide, or hydrogen cyanide; wherein the alkali is lithium, sodium, potassium, or a mixture thereof; wherein the alkaline earth metal is magnesium, calcium, barium, or a mixture thereof; wherein the alkyl is C₁-C₁₂; and wherein the solvent is selected from the group consisting of methanol, ethanol, propanol, isopropanol, butanol, isobutanol, tert-butanol, tetrahydrofuran, dioxane, methoxyethanol, ethoxyethanol, and a mixture thereof.

3. The process according to claim 1, wherein the intermediate of formula (II) is prepared by reacting a Schiff base of the following formula: 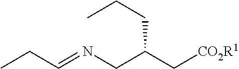
with a source of cyanide in an aqueous solution and optionally in the presence of a solvent, wherein the source of cyanide is alkali cyanide, alkaline earth metal cyanide, zinc cyanide, or hydrogen cyanide; and wherein R¹ is selected from the group consisting of hydrogen, alkali, alkaline earth metal, alkyl, and a mixture thereof; wherein the source of cyanide is alkali cyanide, alkaline earth metal cyanide, zinc cyanide, or hydrogen cyanide; wherein the alkali is lithium, sodium, potassium, or a mixture thereof; wherein the alkaline earth metal is magnesium, calcium, barium, or a mixture thereof; wherein the alkyl is C₁-C₁₂; and wherein the solvent is selected from the group consisting of methanol, ethanol, propanol, isopropanol, butanol, isobutanol, tert-butanol, tetrahydrofuran, dioxane, methoxyethanol, ethoxyethanol, and a mixture thereof.

4. The process according to claim 1, wherein the intermediate of formula (II) is prepared by reacting the cyanohydrin of propionaldehyde of the following formula: 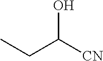
with a primary amine of the following formula: 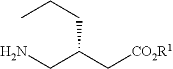
in an aqueous solution and optionally in the presence of a solvent, wherein R¹ is selected from the group consisting of hydrogen, alkali, alkaline earth metal, alkyl, and a mixture thereof; wherein the source of cyanide is alkali cyanide, alkaline earth metal cyanide, zinc cyanide, or hydrogen cyanide; wherein the alkali is lithium, sodium, potassium, or a mixture thereof; wherein the alkaline earth metal is magnesium, calcium, barium, or a mixture thereof; wherein the alkyl is C₁-C₁₂; and wherein the solvent is selected from the group consisting of methanol, ethanol, propanol, isopropanol, butanol, isobutanol, tert-butanol, tetrahydrofuran, dioxane, methoxyethanol, ethoxyethanol, and a mixture thereof.

5. The process according to claim 1, wherein the process of converting the intermediate of formula (II) to the intermediate of formula (IX) further comprises the steps of:
(1) hydrolyzing the intermediate of formula (II) to an intermediate of formula (III) 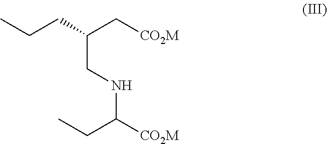
wherein M is an alkali or an alkaline earth metal or their mixture thereof, wherein the alkali is lithium, sodium, potassium, or a mixture; and wherein the alkaline earth metal is magnesium, calcium, barium, or a mixture thereof,
(2) reacting the intermediate of formula (III) with an acid to form an intermediate of formula (IV): 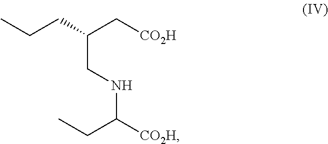 [SMILES: CCC[C@@H](CNC(CC)C(=O)O)CC(=O)O]
and
(3) converting the intermediate of formula (IV) to the intermediate of formula (IX).

6. The process according to claim 1, wherein the compound of formula (IX) is resolved with dehydroabietylamine.

7. The process according to claim 1, wherein the compound of formula (IX) is resolved with an alkaline protease.

8. A compound of the formula:

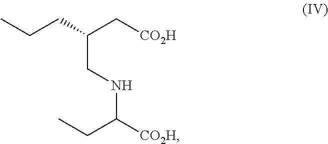 [SMILES: CCC[C@@H](CNC(CC)C(=O)O)CC(=O)O]

9. A salt of the formula:

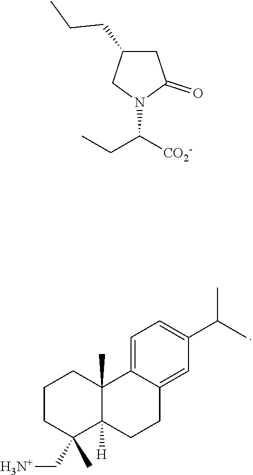

10. A compound of the formula: 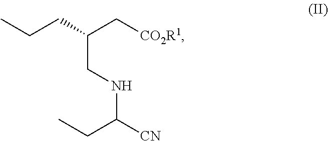
wherein R¹ is hydrogen, alkyl, alkali, or an alkaline earth metal; wherein the alkali is lithium, sodium, potassium or a mixture thereof; and wherein the alkaline earth metal is magnesium, calcium, barium, or a mixture thereof.

11. A compound of the formula: 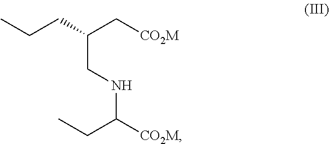
wherein M is an alkali or an alkaline earth metal; wherein the alkali is lithium, sodium, potassium or a mixture thereof; and wherein the alkaline earth metal is magnesium, calcium, barium, or a mixture thereof.

12. A compound of the formula: 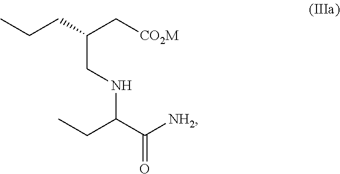
wherein M is an alkali or an alkaline earth metal; wherein the alkali is lithium, sodium, potassium or a mixture thereof; and wherein the alkaline earth metal is magnesium, calcium, barium, or a mixture thereof.
